(19) United States
(12) Patent Application Publication
(10) Pub. No.: US 2015/0224643 A1
(43) Pub. Date: Aug. 13, 2015

(54) OPERATOR-CONTROLLED ELEMENT FOR A POSITIONING DEVICE OF A MEDICAL APPARATUS AND MEDICAL APPARATUS INCORPORATING SAID OPERATOR-CONTROLLED ELEMENT

(71) Applicant: Carl Zeiss Meditec AG, Jena (DE)

(72) Inventors: Stefan Ernsperger, Ellwangen (DE); Frank Rudolph, Aalen (DE)

(21) Appl. No.: 14/621,168

(22) Filed: Feb. 12, 2015

(30) Foreign Application Priority Data
Feb. 12, 2014 (DE) 10 2014 001 784.2

(51) Int. Cl. B25J 9/16

(52) U.S. Cl. CPC B25J 9/161; A61B 2019/2269

(57) ABSTRACT

The invention is directed to an operator-controlled element for a positioning device of a medical apparatus for operation by the mouth of an operator. The operator-controlled element includes a main body and a first sensor for receiving a first operator signal that can be triggered by the operator with the mouth. The operator-controlled element includes a control unit, which is configured to generate a first control signal for a first drive of the positioning device depending on the first operator signal. The invention further relates to a medical apparatus with such an operator-controlled element.

### CROSS REFERENCE TO RELATED APPLICATION

This application claims priority of German patent application no. 10 2014 001 784.2, filed Feb. 12, 2014, the entire content of which is incorporated herein by reference.

### FIELD OF THE INVENTION

The invention relates to an operator-controlled element for a positioning device of a medical apparatus for operation by the mouth of an operator. The operator-controlled element includes a main body and has a first sensor for receiving a first operator signal that can be triggered by the operator with the mouth. The invention further relates to a medical apparatus with a medical instrument, and with a positioning device on which the medical instrument is mounted. The positioning device has at least one degree of freedom of movement and at least one drive for adjusting or supporting a position of the medical instrument along the at least one degree of freedom of movement.

### BACKGROUND OF THE INVENTION

Various medical apparatuses are generally used during a surgical procedure. The course of the surgical procedure is frequently interrupted or delayed by operating procedures that have to be performed on the medical apparatuses during the surgery. This has the result that the overall duration of the surgery is prolonged. This is especially the case when the medical apparatus is operated manually by the operating physician himself. To actuate the medical apparatus, the physician has to withdraw his hand from the field of surgery and, if necessary, put to one side any appliances that have been held in the hand. Thereafter, when resuming the surgery, he has to align his hand and the appliances all over again, which prolongs the surgical procedure.

It is known from the prior art to provide medical apparatuses with foot switches as operator-controlled elements, so that the operating physician can have his hands free during the treatment. Such a foot switch is disclosed in United States patent application publication 2006/0210277, for example. However, a disadvantage of such foot switches is that the stability of the operating physician is jeopardized, particularly if he is standing during the surgery, since the foot switches require a shift of gravity on the standing leg of the physician. This makes the guiding of medical instruments more difficult for the physician.

U.S. Pat. No. 7,375,880 discloses a further medical apparatus, which comprises a surgical microscope with a mouth switch and a stand. By biting on the mouth switch, a contact is closed, so that a current flows, as a result of which a locking mechanism in a pivot joint of the stand is released. The surgical microscope can then be spatially shifted by the physician applying forces to the mouthpieces. A disadvantage of this arrangement is that the forces for moving the surgical microscope have to be transmitted directly by mouth by the operating physician, which can lead to straining in particular of the muscles of the face, neck and throat.

### SUMMARY OF THE INVENTION

An object of the present invention is to make available an operator-controlled element for a positioning device of a medical apparatus, which as far as possible permits ergonomic and smooth positioning without the assistance of the hands.

The operator-controlled element of the invention is for a positioning device of a medical apparatus with the control thereof being by the mouth of the operator. The positioning device includes a first drive and the operator-controlled element includes: a base body; an operator unit configured to permit the operator to act thereupon with the mouth to generate a first operator-control signal; a first sensor for receiving the first operator-control signal; and, a control unit configured to generate a first control signal for the first drive in dependence upon the first operator-control signal.

According to a feature of the invention, the operator-controlled element includes a control unit which is configured to generate a first control signal for a first drive of the positioning device in dependence upon the first operator signal.

The first sensor of the operator-controlled element is configured to receive a first operator signal that is triggered by an operator with the mouth. Depending on the first operator signal, a first control signal is generated in the control unit, which control signal is suitable for controlling a first drive of the positioning device. In this way, by actuating the first sensor by mouth, a change of position of the medical apparatus in one degree of freedom of movement of the positioning device can be actively brought about or supported. The control unit can be integrated in the operator-controlled element or configured separately or can be configured as part of a higher-level control unit of the medical apparatus. Terms which in the context of this invention contain the word “control” (such as “control unit”, “control signal” or “controlling”) are not limited to traditional controls and instead can also comprise closed-loop controls.

In one embodiment of the invention, the operator-controlled element has a second sensor, which is configured to receive a second operator signal that can be triggered by the operator with the mouth and that differs from the first operator signal, and the control unit is configured to generate a second control signal for a second drive of the positioning device depending on the second operator signal. The first sensor and the second sensor can be realized in a common sensor element or as two separate sensors. It is important here that two different operator signals can be received by means of the common sensor element or by means of the first sensor and the second sensor, which operator signals preferably differ from each other in terms of the spatial directions in which they are triggered. By actuating the second sensor with the mouth, a change of position of the medical apparatus can be actively brought about or supported by controlling a further drive of the positioning device in a further degree of freedom of movement.

In further embodiments of the invention, the first sensor and/or the second sensor are configured as force or moment sensors for measuring a force or a moment, in particular a bending moment. Thus, particularly simple and robust sensors are available for implementation of the inventive concept.

In a further embodiment of the invention, a bite strip is present which is mounted so as to be movable in a first direction relative to the main or base body, and the first sensor is configured as a position sensor for measuring an excursion or deflection of the bite strip in the first direction relative to the main body. This permits a particularly intuitive operation of the medical apparatus, since the apparatus can be configured in such a way that the first control signal for the first drive leads to a movement of the medical appliance on the positioning device parallel to the excursion of the bite strip in the first direction.

In a further embodiment of the invention, the bite strip is mounted so as to be movable in a second direction relative to the main body, and the second sensor is configured as a position sensor for measuring an excursion or deflection of the bite strip in the second direction relative to the main body. During operation, therefore, the bite strip can be moved by the operator in a second direction, in particular different from the first direction, relative to the main body of the operating unit. This further improves the intuitive operation of the medical apparatus.

In a further embodiment of the invention, a third sensor is present, which is configured to receive a third operator signal that can be triggered by the operator with the mouth, and the control unit is configured to generate a signal for controlling one or more brakes of the positioning device depending on the third operator signal. In particular, a locking of one or more degrees of freedom of movement of the positioning device can be set up or released with the aid of the third sensor.

In a further embodiment of the invention, the operator-controlled element comprises an attachment device for securing the main or base body on the medical apparatus. After the operator-controlled element has been secured on the medical apparatus, it is thus possible for a movement of a medical instrument mounted on the positioning device, which movement takes place along a degree of freedom of movement and is generated or supported by a drive of the positioning device, to be supported by the operator applying muscle force to the operator-controlled element. In this way, a movement of the medical apparatus can be accelerated on the positioning device when passing from one work position to another work position and/or the drives of the positioning device can be of lower power.

A further object of the present invention is to provide a medical apparatus having an operator-controlled element.

The medical apparatus of the invention includes: a positioning device; a medical instrument held by the positioning device; the positioning device having at least one degree of freedom of movement and having at least one drive for adjusting or supporting a position of the medical instrument along the at least one degree of freedom; an operator-controlled element for the positioning device with the control thereof being via the mouth of the operator; the positioning device including a first drive; and, the operator-controlled element including: a base body; an operator unit configured to permit the operator to act thereupon with the mouth to generate a first operator-control signal; a first sensor for receiving the first operator-control signal; and, a control unit configured to generate a first control signal for the first drive in dependence upon the first operator-control signal.

### BRIEF DESCRIPTION OF THE DRAWINGS

The invention will now be described with reference to the drawings wherein:

FIG. 1 shows an illustrative embodiment of a medical apparatus according to the invention, and

FIG. 2 shows various illustrative embodiments of operator-controlled elements according to the invention.

### DESCRIPTION OF THE PREFERRED EMBODIMENTS OF THE INVENTION

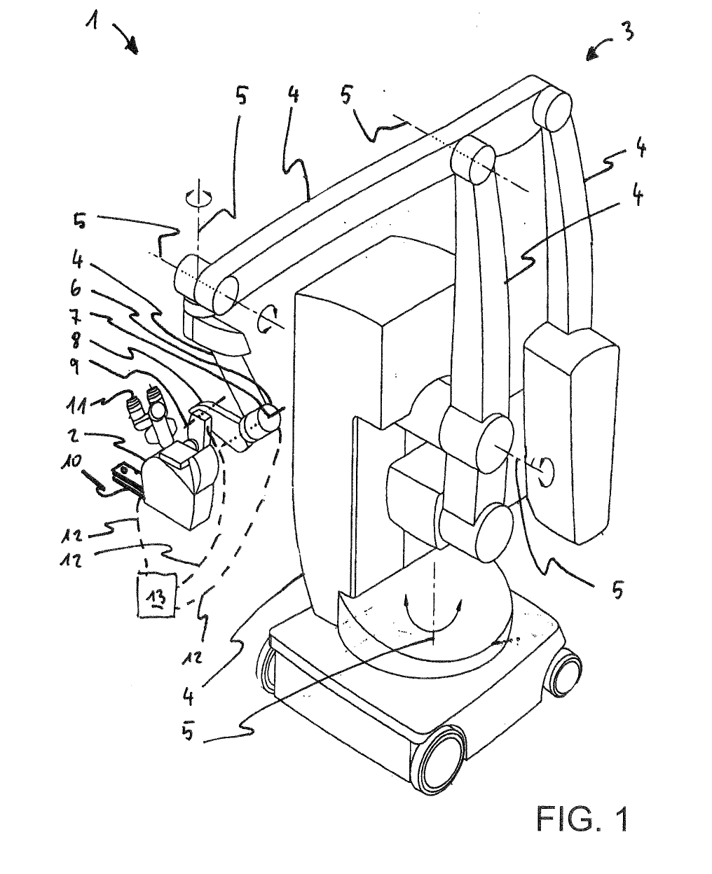

In FIG. 1, a medical apparatus 1 according to the invention is shown which includes a medical instrument in the form of a surgical microscope 2 that is mounted on a positioning device in the form of a stand 3.

In this illustrative embodiment, the stand 3 is constructed from a plurality of support arms 4 which are arranged in series and in parallel and are interconnected via joint connections 5. A first pivot joint 7 is assigned a first drive 6, and a second pivot joint 9 is assigned a second drive 8. With the first drive 6, a torque can be generated about a rotation axis of the first pivot joint 7, such that an angle travel between the support arms that are interconnected by the first pivot joint 7 can be adjusted. Correspondingly, with the second drive 8, a torque can be generated about a rotation axis of the second pivot joint 9, such that an angle excursion or deflection between the support arms that are interconnected by the second pivot joint 9 can be adjusted.

An operator-controlled element 10 is mounted on the surgical microscope 2 and can be gripped in the mouth by an operator. Here, the word “mouth” is to be understood as meaning all body parts in and around an oral cavity of the operator, for example teeth, tongue, gums, jaws or lips. In this illustrative embodiment, the operator-controlled element 10 is configured to be held and operated by the operator using the teeth.

The operator-controlled element 10 is arranged on the surgical microscope in such a way that the operator can use his eyes to observe a field of surgery through eyepieces 11 of the surgical microscope 2 while holding the operator-controlled element by the teeth or by another part of the mouth.

With the aid of the operator-controlled element 10, the operator is able to adjust a position of the surgical microscope 2 in at least one degree of freedom. The basic function is described below by using the example of a pivoting movement about a rotation axis of the first pivot joint 7. For this purpose, as is described in more detail below, the operator uses his mouth to apply a first operator signal to a first sensor of the operator-controlled element 10. The first operator signal is transmitted to a control unit 13 via a signal transmission line 12, for example a cable, a printed circuit board line, or a wireless connection. In the control unit 13, a first control signal is determined on the basis of the first operator signal, and this first control signal is transmitted via a further signal transmission line 12 to the first drive 6 of the stand. The drive 6 is assigned to the first pivot joint 7. A torque about the rotation axis is adjusted via the first drive 6, such that the surgical microscope 2 is swiveled about the rotation axis to the desired position.

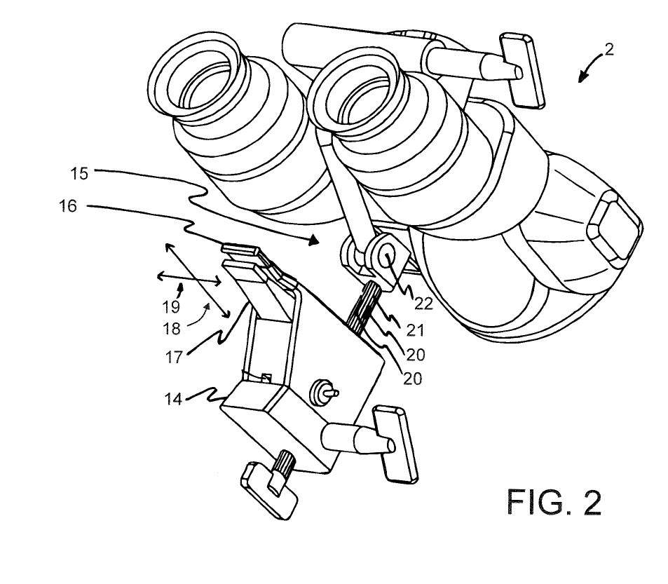

An illustrative embodiment of an operator-controlled element 10 according to the invention is shown in greater detail in FIG. 2.

The operator-controlled element 10 includes a main or base body 14, which is attached to the surgical microscope 2 via an attachment device 15. A first bite strip 16 is formed on the main body 14. The bite strip 16 is rigidly connected to the main body. The operator is able to bite with the teeth of one jaw (in this case the upper jaw) on the bite strip.

A second bite strip 17 is arranged underneath the first bite strip 16 and, in this embodiment, is mounted so as to be movable relative to the main body 14 in two directions along the double arrows (18, 19). During use, the second bite strip 17 is in contact with the teeth of the other jaw of the operator (in this case the lower jaw). By moving the lower jaw relative to the upper jaw, the operator can move the second bite strip 17 in the direction of the two arrows (18, 19).

With the aid of a first sensor not shown in detail in FIG. 2, a first operator signal is determined in the form of an excursion or deflection of the second bite strip 17 in the direction of the first double arrow 18. A second sensor, likewise not shown in detail, serves to determine a second operator signal in the form of an excursion of the second bite strip 17 in the direction of the second double arrow 19. The first sensor and the second sensor are preferably configured as position sensors which, in the context of this invention, are to be understood as both simple switches, which are switched starting from a defined excursion of the second bite strip 17, and also sensors for measuring an excursion distance, an excursion speed, an excursion acceleration, or another physical parameter suitable for direct or indirect determination of an excursion.

The invention has been explained above on the basis of a second bite strip that is mounted so as to move two ways in translation relative to the main body. However, it is not limited to such an embodiment. Without departing from the scope of the invention, the bite strip can be mounted so as to be movable relative to the main body both in translation (as a slide joint) and also in rotation (as a pivot joint), or in a combination of translation and rotation (as a pivot/slide joint). It is also entirely conceivable for the second bite strip to be configured with just a single bearing or with more than two bearings relative to the main body. The number and nature of the sensors are to be suitably adapted to the form of the bearing.

A further illustrative embodiment of an operator-controlled element 10 according to the invention is explained below with reference to FIG. 2. Here, the second bite strip 17 is also rigidly connected to the main body 14. Alternatively, in this illustrative embodiment, the second bite strip can also be omitted, such that the operator only grips the first bite strip 16 in the mouth. In this illustrative embodiment, instead of position sensors for measuring an excursion of the second bite strip, two sensors in the form of strain gauges 20 for measuring bending stresses are arranged on a securing rod 21 of the attachment device 15. During use, the operator uses his mouth to grip the main body via the bite strip and exerts a force and/or a torque on the main body. The force and/or the torque leads to a slight deformation or bending of the securing rod, which is detected as an operator signal via the strain gauges 20. As in the first illustrative embodiment, the operator signal is transmitted to the control unit, in which a control signal for the drives is generated on the basis of the operator signal. The operator-controlled element can be equipped with one or more strain gauges for measuring one or more operator signals.

Without restricting the generality of the invention, it is also possible, instead of the above-described strain gauges, to use other force or moment sensors for direct or indirect measurement of a force or pressure or moment applied to the main body by the operator via an operator-controlled element according to the invention.

A third illustrative embodiment of an operator-controlled element according to the invention is likewise described with reference to FIG. 2. The third illustrative embodiment differs from the first illustrative embodiment in that the position sensors are configured as rotation or tilt sensors in a joint 22 of the attachment device 15. During use of the operator-controlled element, the position sensors are thus not located in the oral cavity of the operator, as they are in the first illustrative embodiment, but instead outside the oral cavity.

The invention has been described above on the basis of two drives on two pivot joints of the stand. However, it is not limited to such an arrangement. Without departing from the scope of the present invention, any kinds of joint connections (for example slide joints, pivot/slide joints or screw joints) can be provided between support arms of the stand, and any combinations of drives on the joint connections of the stand. It is important that at least one joint connection of the stand is assigned a drive with the aid of which a torque can be applied about a rotation axis or a force can be applied in the direction of a joint axis, such that it is possible to adjust an excursion (for example an angle or a distance) between the support arms that are interconnected by the joint connection.

The stand 3 is preferably constructed in such a way that the surgical microscope 2 can be oriented in six degrees of freedom (three rotary degrees of freedom and three translatory degrees of freedom) within a work area. However, without restricting the generality of the invention, embodiments of the stand having fewer or more than six degrees of freedom are also conceivable.

In all the illustrative embodiments, a third sensor in the form of a switch can be provided, which is configured to receive a third operator signal that can be generated by the teeth, lips or other parts of the mouth of the operator. The third operator signal is likewise transmitted to the control unit 13 and is there converted into a signal or into several signals for locking or releasing a brake or several brakes in the joint connections (5, 7, 8) of the stand 3. This ensures that the surgical microscope 2 is securely fixed in a work position. The third sensor can preferably be used to release those brakes of the stand 3 that are assigned to the drives (6, 8) of the stand, which can be controlled via the first sensor and the second sensor (and, if appropriate, further sensors) of the operator-controlled element 10. In this way, when moving the surgical microscope 2 from one work position to another, an operator can firstly release the brakes by actuating the third sensor and can then move the surgical microscope 2 to the new work position, without great physical effort, by actuating the first sensor and/or the second sensor and/or optionally further sensors.

In a further illustrative embodiment not shown, the stand with the drives is configured in such a way that, in the first instance, only rotary angles of the surgical microscope can be adjusted with the aid of the sensors of the operator-controlled element and of the drives. Translatory positions of the surgical microscope in the work area can be adjusted either manually or in a conventional way supported by a motor. This embodiment takes account of the fact that, in many operating situations, the surgical microscope is moved only to a slight extent in translation and to a much greater extent in rotation.

In yet another illustrative embodiment not shown here, the drives are dimensioned in such a way that their power alone is not sufficient to move the surgical microscope in the direction of a new work position starting from all the work positions of the surgical microscope in the work area. In this case, the drives merely assist the operator when moving the surgical microscope from one work position to another.

The invention has been described above on the basis of operator-controlled elements that are rigidly connected to a surgical microscope. However, without restricting the generality of the invention, the operator-controlled element can also be configured separate from the surgical microscope, such that an operator can accommodate it completely or at least partially in the oral cavity. In this case, for example, the signal transmission from the operator-controlled element to the control unit is wireless.

It is understood that the foregoing description is that of the preferred embodiments of the invention and that various changes and modifications may be made thereto without departing from the spirit and scope of the invention as defined in the appended claims.

What is claimed is:

1. An operator-controlled element for a positioning device of a medical apparatus with the control thereof being by the mouth of the operator, the positioning device including a first drive and the operator-controlled element comprising:
a base body;
an operator unit configured to permit the operator to act thereupon with the mouth to generate a first operator-control signal;
a first sensor for receiving said first operator-control signal; and,
a control unit configured to generate a first control signal for said first drive in dependence upon said first operator-control signal.

2. The operator-controlled element of claim 1, the positioning device including a second drive and said operator-controlled element further comprising:
said operator unit being further configured to permit the operator to act thereupon with the mouth to generate a second operator-control signal;
a second sensor for receiving said second operator-control signal; and,
said control unit being further configured to generate a second control signal for said second drive in dependence upon said second operator-control signal.

3. The operator-controlled element of claim 1, wherein said first sensor is configured as one of a force sensor and torque sensor.

4. The operator-controlled element of claim 2, wherein said second sensor is configured as one of a force sensor and torque sensor.

5. The operator-controlled element of claim 1, wherein said operator unit is configured as a bite strip mounted so as to be movable in a first direction relative to said base body; and, said first sensor is configured as a position sensor for measuring a deflection of said bite strip in said first direction relative to said base body.

6. The operator-controlled element of claim 2, wherein said operator unit is configured as a bite strip mounted so as to be movable in a first direction relative to said base body; and, said first sensor is configured as a position sensor for measuring a deflection of said bite strip in said first direction relative to said base body.

7. The operator-controlled element of claim 6, wherein said bite strip is mounted so as to be movable also in a second direction relative to said base body; and, said second sensor is configured as a position sensor for measuring a deflection of said bite strip in said second direction relative to said base body.

8. The operator-controlled element of claim 2, wherein said positioning device includes one or more brakes and said operator-controlled element further comprises:
said operator unit being further configured to permit the operator to act thereupon with the mouth to generate a third operator-control signal;
a third sensor for receiving said third operator-control signal; and,
said control unit being further configured to generate a signal for driving said one or more brakes in dependence upon said third operator-control signal.

9. The operator-controlled element of claim 1, further comprising an attachment device for attaching said base body to said medical apparatus.

10. A medical apparatus comprising:
a positioning device;
a medical instrument held by said positioning device;
said positioning device having at least one degree of freedom of movement and having at least one drive for adjusting or supporting a position of said medical instrument along said at least one degree of freedom;
an operator-controlled element for said positioning device with the control thereof being via the mouth of the operator;
said positioning device including a first drive; and,
said operator-controlled element including: a base body; an operator unit configured to permit the operator to act thereupon with the mouth to generate a first operator-control signal; a first sensor for receiving said first operator-control signal; and, a control unit configured to generate a first control signal for said first drive in dependence upon said first operator-control signal.

11. The medical apparatus of claim 10, wherein said positioning device includes a second drive and said operator-controlled element further includes:
said operator unit being further configured to permit the operator to act thereupon with the mouth to generate a second operator-control signal;
a second sensor for receiving said second operator-control signal; and,
said control unit being further configured to generate a second control signal for said second drive in dependence upon said second operator-control signal.

12. The medical apparatus of claim 10, wherein said first sensor is configured as one of a force sensor and torque sensor.

13. The medical apparatus element of claim 11, wherein said second sensor is configured as one of a force sensor and torque sensor.

14. The medical apparatus of claim 10, wherein said operator unit is configured as a bite strip mounted so as to be movable in a first direction relative to said base body; and, said first sensor is configured as a position sensor for measuring a deflection of said bite strip in said first direction relative to said base body.

15. The medical apparatus of claim 11, wherein said operator unit is configured as a bite strip mounted so as to be movable in a first direction relative to said base body; and, said first sensor is configured as a position sensor for measuring a deflection of said bite strip in said first direction relative to said base body.

16. The medical apparatus of claim 15, wherein said bite strip is mounted so as to be movable also in a second direction relative to said base body; and, said second sensor is configured as a position sensor for measuring a deflection of said bite strip in said second direction relative to said base body.

17. The medical apparatus of claim 11, wherein said positioning device includes one or more brakes and said operator-controlled element further comprises:
said operator unit being further configured to permit the operator to act thereupon with the mouth to generate a third operator-control signal;
a third sensor for receiving said third operator-control signal; and,
said control unit being further configured to generate a signal for driving said one or more brakes in dependence upon said third operator-control signal.

18. The medical apparatus of claim 10, further comprising an attachment device for attaching said base body to said medical apparatus.
